(19) United States
(12) Patent Application Publication
(10) Pub. No.: US 2022/0354763 A1
(43) Pub. Date: Nov. 10, 2022

(54) COMPOSITION FOR COSMETIC TATTOO LIGHTENING AND METHOD OF USING SAME

(71) Applicant: Yaroslava ARTYOMENKO, Alpharetta, GA (US)

(72) Inventor: Yaroslava ARTYOMENKO, Alpharetta, GA (US)

(21) Appl. No.: 17/738,087

(22) Filed: May 6, 2022

(30) Foreign Application Priority Data
May 6, 2021 (LB) 12203

(51) Int. Cl. A61K 8/41; A61K 8/27; A61K 8/25; A61K 8/73; A61K 8/34; A61Q 19/02

(52) U.S. Cl. CPC A61K 8/41; A61K 8/27; A61K 8/25; A61K 8/731; A61K 8/345; A61Q 19/02; A61K 2800/5922

(57) ABSTRACT

An enhanced alkaline pigment lightening suspension formulation for fading of unwanted cosmetic tattoos is proposed. The improved formulation of the claimed chemical tattoo lightening suspension is additionally complemented with a suggested neutralizing technique. The proposed innovative method includes the application of alkaline solution followed by an acid solution to reduce side effects. Additionally, a unique method of administration of the post-removal skin rehabilitation is proposed. The treatment is proven effective in scar reduction and consists of the administration of percutaneous collagen induction treatments following the aforementioned tattoo lightening treatments.

### FIELD OF INVENTION

A novel composition and method for cosmetic and artistic tattooing. The method of use includes:
Step 1: Topical application of the alkali suspension
Step 2: Topical application of acidic neutralization
Step 3: Percutaneous collagen induction treatment

### BACKGROUND OF THE INVENTION

Tattooing is a technique of inserting ink into the skin via a needle using an electric tattoo-machine. The art of tattooing has gained extraordinary popularity nowadays. In particular, cosmetic tattoos or so-called Permanent Makeup, has become a part of contemporary fashion. However, in a manuscript released by the U.S. Food and Drug Administration (FDA) on Aug. 24, 2020 titled “Tattoos & Permanent Makeup: Fact Sheet”, the FDA expressed concern not only about the quality of pigments being used by technicians worldwide, but also about a commonly experienced problem among tattoo owners—dissatisfaction. The document mentioned “Despite advances in laser technology, removing a tattoo is a painstaking process, usually involving several treatments and considerable expense. Complete removal without scarring may be impossible.”

Current treatment options for tattoo removal include a variety of techniques, including lasers, dermabrasion, salabrasion, surgical excision, cryotherapy, as well as various saline, acidic and alkali compositions, each more or less effective on particular types/colors of pigments. A wide range of pigments are used in tattoos, from inorganic materials like titanium dioxide and iron oxides to organic materials such as carbon black, azo dyes, acridine, quinoline, phthalocyanine and naphthol derivates, and other mixtures.

The biokinetics of tattoo inks therefore underlie different phases and mechanisms. Firstly, an immediate passive distribution of particles and soluble substances will occur at the time of the tattooing procedure. Secondly, phagocytic cells will actively translocate particles to the draining lymph nodes until wound healing is complete. Finally, and over an extended course of time, cell metabolism and sunlight exposure degrade the compounds within the pigments and residual large polymers. Laser removal procedures attempted on the tattooed area have a similar effect as well. Clinical studies show that upon laser irradiation, all organic pigments are cleaved into benzene and hydrogen cyanide-carcinogenic and sensitizing compounds. Many tattoo inks are a mixture of colors with a wide range of tints (white, black, blue, green, violet, orange) and are difficult to classify as a single pigment. When a color is a mixture of two or more pigments (one of which is reflective, the other absorptive), upon laser irradiation, some of the reflected light from non-absorbing pigment reaches the absorbing pigment, causing plasma formation. The resulting reaction not only disrupts dermal cells, but also affects physical properties of both pigments.

These reactions are why cosmetic tattoo (permanent make-up) removal is an aesthetically sensitive procedure. Experience has shown that laser irradiation oftentimes triggers a conversion of red shades of lip pigments into purple. White titanium oxide (an ingredient present in most inks to regulate pigment shades) also undergoes a color change to bluish or greenish tints. Scientists estimate that this phenomenon of laser-induced color change of inorganic pigments into darker shades possibly occurs due to iron oxide and titanium oxide being transferred into a different state of oxidation.

On the other hand, attempts to remove organic pigments with lasers, at times, results in their transformation into unexpectedly bright colors, unacceptable to be worn on the face. From observations made in some publications, a carbonization reaction of the organic pigment also appears to be plausible. See FIG. 1.

### TATTOO-PIGMENT DISTRIBUTION IN HUMAN SKIN

A needle penetrates the skin through the epidermis delivering ink into the papillary layer of the dermis, where pigment particles accumulate. As with any type of trauma to the dermis, the skin tissue swells (edema) allowing migration of immune system cells (neutrophils and macrophages) to the wound site in order to phagocytose foreign substances (the pigment inserted), cell debris, and microbes. Any damaged collagen in the wounded papillary dermis is then repaired through the action of fibroblasts, ultimately laying down scar tissue. Eventually, dermal cells (typically fibroblasts or macrophages) engulf the pigment particles. Once the pigment particles have been engulfed, they usually reside within the cell cytoplasm. That is how tattooed pigment stays permanently in the skin. Over long periods of time the tattoo ink particles can be found to gradually move to the deeper dermis (i.e., reticular dermis), which gives the tattoo a faded and blurred appearance. Importantly, after tattoo ink insertion, and in cases of laser irradiation removal attempts, associated pigment particles can be found to leave the skin via its vasculature and enter the lymphatic system (nodes) . This particular property leads to a common side-effect in cosmetic tattoo of the eyebrows. Engulfed with collagen and submerged into deeper skin layers, brown pigments become perceptible as gray, blue, or purple. The distorted color-perception of the originally introduced pigment is described in physics as the Tyndall effect: “In turbid environments, violet and blue light is scattered the most, and orange and red are the weakest.” See FIG. 2.

The need for an alternative to laser treatment for facial cosmetic tattoos fading has spurred multiple experiments to improve chemical removal methods. With all the above mention in view, this invention consists in developing an optimal composition for minimally traumatic lightening of undesirable facial cosmetic tattoos. and to construct method of application in combination with a well-studied post-procedural care.

There are three major kinds of chemical tattoo fading products existing on the world market: saline, acidic and alkaline. The present invention is a composition of alkaline nature.

There are three major kinds of chemical tattoo fading products existing on the world market: saline, acidic and alkaline. The present invention is a composition of alkaline nature.

### The Disadvantages of the Similar Products Existing on the World Market

Referring to Chemical Composition and Physical Properties:

Instability of the suspension throughout time. Mineral oxides used in alkaline compositions tend to precipitate and stick together, which results in a waste of approximately ¼ of the product purchased by the technician.

The consistency of similar products is either too thick or too thin, which, in former case, creates difficulty in application and, in the latter case, a danger of accidental distribution of the product to locations not indicated for treatment, such as the eyes or mouth.

Referring to the Method of Application:

All similar alkaline products are applied in a variety of techniques to the skin and have proven effective on fading pigments. Nevertheless, there are multiple cases of complications occurring as a result of complete disregard of alkali properties and scientific data about its reaction with skin proteins. Clinical trials of the alkaline injury model showed that after inflicting alkaline injury to the skin of rats, the subcutaneous tissue pH reached its peak value at the 32nd minute post-application and did not recover to the pre-experimental levels even when measured 90 minutes later (Yano, Hata, Matsuka, Ito, & Matsuda 1993). Despite this knowledge, some manufacturers suggest a prolonged application of such compositions (as long as 24 hours), which results in severe damage of the treated skin area and consequent scarring.

Absence of aftercare suggestions and manual for the individuals completing the tattoo fading procedure.

A lack of comprehensive theoretical base for educating technicians purchasing and applying the product.

Bearing in mind all the existing shortcomings in the current market of alkaline tattoo-fading products, the following improvements were implemented.

### SUMMARY

A composition for fading of cosmetic tattoo, comprising: isopropanol, triethanolamine, Calcium oxide, Zinc oxide, Magnesium Oxide, propylene glycol, silicon dioxide, ethyl cellulose and deionized water. The composition preferrably consists of (by weight): 27.15% of deionized water, 56.6% isopropanol, 3% of triethanolamine, 7% of Calcium oxide, 1% of Zinc oxide, 0.5% of Magnesium Oxide, 4% of propylene glycol, 0.5% silicon dioxide, 0.25% ethyl cellulose.

Also the method of using composition comprising: isopropanol, triethanolamine, Calcium oxide, Zinc oxide, Magnesium Oxide, propylene glycol, silicon dioxide, ethyl cellulose and deionized water is proposed for neutralization of fading suspension, systematic lightening of cosmetic tattoo and rehabilitation of skin scarring due to cosmetic tattoo application or/and removal attempts.

### BRIEF DESCRIPTION OF THE DRAWINGS:

FIG. 1 Laser-induced color transformation in cosmetic tattoo;

FIG. 2 The pigment covered with layers of collagen, and the tissue is perceived as cold-hued due to short wavelength scattering of light;

FIG. 3 From Left-to-right: Example of correction of an undesirably bright color of lip tattoo; Example of correction of deep-skin eyebrow tattoo application;

FIG. 4 From Left-to-right: Lightening of the two years old cosmetic eyebrow tattoo; Lightening of the six years old cosmetic eyebrow tattoo;

FIG. 5 Extraction of pigment substance from the excess of collagen formed above its surface due to unsuccessful laser tattoo removal, and reducing scarring formed as a result of aggressive application of cosmetic tattoo: lightening of lip cosmetic tattoo (after traumatic laser removal attempts) with further scar reduction applying REVITAL technique and new cosmetic tattoo application, and the reduction of scars formed as a result of aggressive eyebrow cosmetic tattoo application.

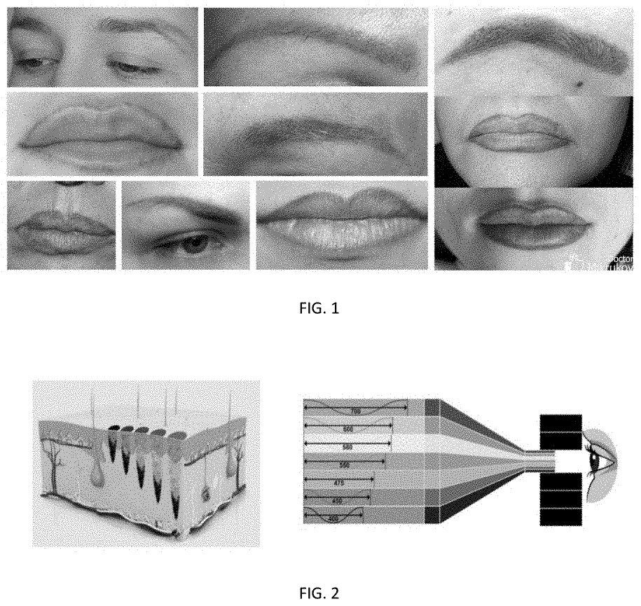

### DETAILED DESCRIPTION OF THE PREFERRED EMBODIMENT

### Ingredients Included in the Proposed Composition

| | CAS | | | | Mass |
|---|---|---|---|---|---|
| IUPAC of | Registry | | | | (g) per |
| the ingredient | number | EC code | Molecular formula | Percentage | 1 L |
| Deionized Water | 7732-18-5 | 231-79-2 | H2O | 27.15 | 271.5 |
| Isopropanol | 67-63-0 | 200-661-7 | C3H8O | 56.6 | 566 |
| Triethanolamine | 102-71-6 | 203-049-8 | (HOCH2CH2)3N | 3 | 30 |
| Calcium Oxide | 1305-78-8 | 215-138-9 | CaO | 7 | 70 |
| Zinc Oxide | 1314-13-2 | 215-222-5 | ZnO | 1 | 10 |
| Magnesium Oxide | 1309-48-4 | 215-171-9 | MgO | 0.5 | 5 |
| Propylene Glycol | 57-55-6 | 200-338-0 | C3H8O2 | 4 | 40 |
| Silicon Dioxide | 7631-86-9 | 231-545-4 | SiO2 | 0.5 | 5 |
| Ethyl Cellulose | 9004-57-3 | 618-384-9 | C6H7O2(OR1)(OR2) | 0.25 | 2.5 |
| | | | *where R1 and R2 may be | | |
| | | | any of the following: | | |
| | | | —H or —CH2CH3 | | |

### DETAILED DESCRIPTION OF THE PREFERRED EMBODIMENT

### Advantages of the Invented Composition and Method

The Composition Performance Improvement:

A small amount of propylene glycol has been added to keep the products from melting in high heat or from freezing during the avia shipment. Propylene glycol also helps active ingredients of the composition penetrate the epidermis.

Aerosil, or amorphous anhydrous colloidal silicon dioxide (used in pharmaceutical products), was added as a viscosity increasing agent to thicken and thixotropize the suspension and as well as an anti-setting, anti-sagging agent.

Ethyl cellulose (widely used in pharmaceutical compositions) was added as a binding and thickening agent. Its coating properties allow it to serve as a disperser for the mineral compounds used in the composition.

As a result of the abovementioned implemented changes, the suspension obtained has a smooth, creamy texture (suitable for the precise local applications) and is stable in time and impervious to temperature fluctuations during shipments.

The Innovative Method of the Application:

The well-balanced new composition softens collagen fiber and does not require any mechanical application. Topical application for 5-10 minutes (depending on the client's skin texture and thickness) allows softening of the epidermal layers. Wiping the treated area with a water-soaked cotton pad is the second step in the described method.

Clinical studies show that topical treatment of alkaline burns with a weak acid results in more rapid tissue neutralization, reduction of injury, and improved epithelial regeneration. Based on this evidence, the proposed new tattoo-lightening method relies on immediate topical introduction of a mild solution of tricarboxylic acid on the area where the alkaline suspension was previously applied. It is recommended to then keep the treated area moist with the acid solution applied for one minute before wiping dry with a clean cotton pad. This step will assure the neutralization of any residual substance in the treated area and minimize the possibility of side effects.

After the neutralization phase has been completed, treatment with derma pen, using the nano module, is indicated, to peel off the keratinocytes disassembled by the fading composition application.

Dexapanthenol-containing creams are highly effective for skin barrier repair after alkali-induced irritation. An aftercare instruction manual with a list of dexapanthenol-containing pharmaceutical creams is developed and will be suggested to the treated customers.

A clinical study has found a notable increase of collagen and elastin following percutaneous collagen induction therapy. Compared with collagen in the scar tissues, the collagen in the treated area was cross-linked, the stratum corneum was structured normally, and a thickening of the epidermis of about 40% was observed. The method being suggested includes a sequence of follow-up treatments of percutaneous collagen induction therapy to the area previously treated with the tattoo fading suspension to enhance the skin texture.

Through the gathering of clinical data and practical experience, the afore-described method was developed and named “REVITAL”. REVITAL is potentially an effective approach in fading unwanted cosmetic tattoo and reviving the skin to regain normal texture and function.

### EXAMPLES OF USE OF THE INVENTED METHOD AND COMPOSITION

Volunteers seeking tattoo-fading treatment were enrolled in a study that resulted in outcomes described hereafter. The suggested composition and method for fading unwanted cosmetic tattoo is effective for the following needs:

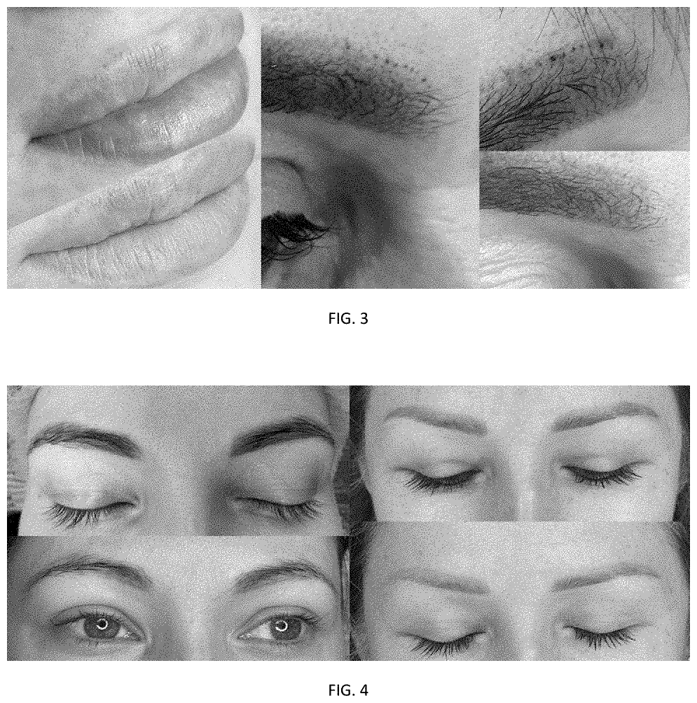

Lightening of fresh (one month since application) cosmetic tattoo and correcting mistakes made by the tattoo artist see in FIG. 3.

Lightening of old cosmetic tattoo; 2-6 years after application. See in FIG. 4.

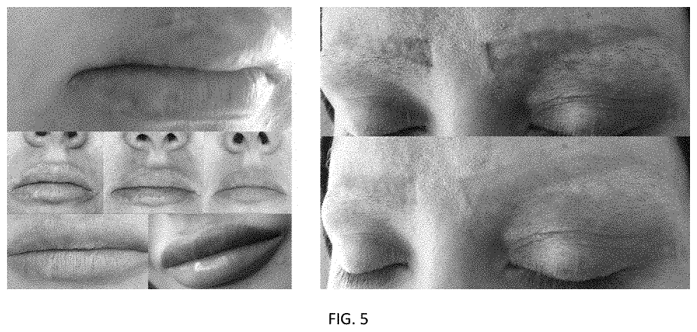

Extraction of pigment substance from the excess of collagen formed above its surface due to unsuccessful laser tattoo removal, and reducing scarring formed as a result of aggressive application of cosmetic tattoo, see FIG. 5.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

What is claimed is:

1. A composition for fading of cosmetic tattoo, comprising: isopropanol, triethanolamine, Calcium oxide, Zinc oxide, Magnesium Oxide, propylene glycol, silicon dioxide, ethyl cellulose and deionized water.

2. The composition of claim 1, wherein the composition consists of (by weight): 27.15% of deionized water, 56.6% isopropanol, 3% of triethanolamine, 7% of Calcium oxide, 1% of Zinc oxide, 0.5% of Magnesium Oxide, 4% of propylene glycol, 0.5% silicon dioxide, 0.25% ethyl cellulose.

3. A method of neutralization of fading suspension, systematic lightening of cosmetic tattoo and rehabilitation of skin scarring due to cosmetic tattoo application or/and removal attempts by using a composition comprising: isopropanol, triethanolamine, Calcium oxide, Zinc oxide, Magnesium Oxide, propylene glycol, silicon dioxide, ethyl cellulose and deionized water.

4. The method of claim 3, wherein the composition consists of (by weight): 27.15% of deionized water, 56.6% isopropanol, 3% of triethanolamine, 7% of Calcium oxide, 1% of Zinc oxide, 0.5% of Magnesium Oxide, 4% of propylene glycol, 0.5% silicon dioxide, 0.25% ethyl cellulose.
